(19) United States
(12) Patent Application Publication
(10) Pub. No.: US 2018/0223356 A1
(43) Pub. Date: Aug. 9, 2018

(54) NANOCHANNEL WITH INTEGRATED TUNNEL GAP

(71) Applicant: ARIZONA BOARD OF REGENTS On behalf of ARIZONA STATE UNIVERSITY, Scottsdale, AZ (US)

(72) Inventors: Brian Alan ASHCROFT, Mesa, AZ (US); Stuart LINDSAY, Phoenix, AZ (US)

(21) Appl. No.: 15/949,406

(22) Filed: Apr. 10, 2018

Related U.S. Application Data
(63) Continuation of application No. 14/971,492, filed on Dec. 16, 2015.
(60) Provisional application No. 62/092,754, filed on Dec. 16, 2014.

(51) Int. Cl. C12Q 1/6869; G01N 33/487

(52) U.S. Cl. CPC C12Q 1/6869; G01N 33/48721

(57) ABSTRACT

A device for electronic sequencing of polymers consisting of a tunnel gap that is self-aligned with a nanochannel.

### CROSS REFERENCE TO RELATED APPLICATIONS

This application is a continuation application of U.S. application Ser. No. 14/971,492, filed Dec. 16, 2015, which claims priority to U.S. provisional application No. 62/092,754 titled “NANOCHANNEL WITH INTEGRATED TUNNEL GAP”, filed Dec. 16, 2014, the entire disclosure of which is incorporated herein by reference in its entirety.

### STATEMENT REGARDING FEDERALLY SPONSORED RESEARCH & DEVELOPMENT

This invention was made with government support under grant number R01 HG006323 awarded by The National Institutes of Health. The government has certain rights in the invention.

### BACKGROUND OF THE INVENTION

In an earlier disclosure “Systems and Devices for Molecule Sensing and Method of Manufacturing Thereof” (US publication no. US 2014-0113386, the '386 publication; see also Pang et al., Pang, Ashcroft et al. 2014, both hereby incorporated by reference) we have described a method of manufacturing a tunnel junction such that individual molecular species give distinct electronic signals when in contact with recognition molecules bound to the electrodes that comprise the tunnel junction. Further described is methodology for cutting a nanopore through the layers that comprise the junction, so that each molecular unit (e.g., DNA base, protein residue or sugar molecule in an oligosaccharide) can be read as it passes the electrodes embedded in the nanopore.

An alternative to passing a polymer through a nanopore is to linearize the polymer by driving the polymer into a long channel with lateral dimensions comparable to the diameter of the polymer. If the polymer is quite stiff, the dimensions of this channel can be quite large, 10× to 50× the diameter of the polymer. So, for DNA, having a diameter of 2 nm, the channel could be up to about 100 nm in width. The persistence length of double stranded DNA is 50 nm, so it is constrained to enter such a channel in a linearized form. In order to read a sequence of the polymer, a small reading device must be placed into the channel, so that the sequence can be read as each base passes the reading device. A system like this has been described by Liang and Chou (Liang and Chou 2008) and it is illustrated in FIG. 1. A channel of tens of nm depth and tens of nm width (but many microns in length) is formed in a glass or silicon substrate 201 (in FIG. 1a). It is coated with a layer of resist 202 that exposes the channel (FIG. 1b). This can be the resist layer that was used to etch the channel in the first place. Metal is then evaporated from an angle 203 so as to partially fill the channel (FIG. 1c). Done from opposite sides of the channel, the channel can then be filled with metal separated by a gap of a few nm (204 FIG. 1d) by stopping the evaporation before the metals on opposite walls contact. Contacts are then made to the metal layers inside the channel lithographically 205 (FIG. 1d) and the device covered with a glass coverslip 206 to seal the channel.

Another method of forming nm sized gaps in metal electrodes is via shadow evaporation of metals is described by Sun et al. (Sun, Chin et al. 2005) and illustrated in FIG. 2. A gold pad 101 is deposited onto a SiO₂ substrate. The SiO₂ is etched away to leave a step 102 at edge of the pad. A second layer of metal is deposited at an angle 103, forming a second pad 104 displace vertically from the first. By controlling the thickness of this second pad, the dimension of a gap between the two pads can be controlled (FIG. 2c).

Perry et al. (Perry and Kandlikar 2006) describe how a nanochannel can be closed by angle deposition of silicon oxide, as illustrated in FIG. 3. A pre-formed nanochannel, shown in cross section 301, is filled with glass deposited at an angle 302. By alternating the angle at which the glass is deposited, the channel becomes filled and covered with a glass layer, leaving a channel with a cross section shown by the dotted line 303.

### BRIEF DESCRIPTION OF THE FIGURES

FIG. 1 illustrates a nanochannel with embedded sensing electrodes according to prior art.

FIG. 2 illustrates a method for making a nanometer scale gap between electrodes by shadow evaporation according to the prior art.

FIG. 3 illustrates a method for capping a pre-existing nanochannel using angle evaporation according to the prior art.

FIG. 4 illustrates two views of a device according to some embodiments of the disclosure.

FIG. 5 illustrates cross-section and plan views of steps, according to some embodiments, in fabrication of a device according to some embodiments of the present disclosure.

FIG. 6 illustrates the sealing and separation of fluid chambers according to some embodiments of the present disclosure.

FIG. 7 illustrates ion-current blockade signals as single stranded DNA molecules pass through the nanochannel and electrode gap in a device according to some embodiments of the present disclosure.

FIG. 8 illustrates tunnel-current signals as single stranded DNA molecules pass through the electrode gap in a device according to some embodiments of the present disclosure.

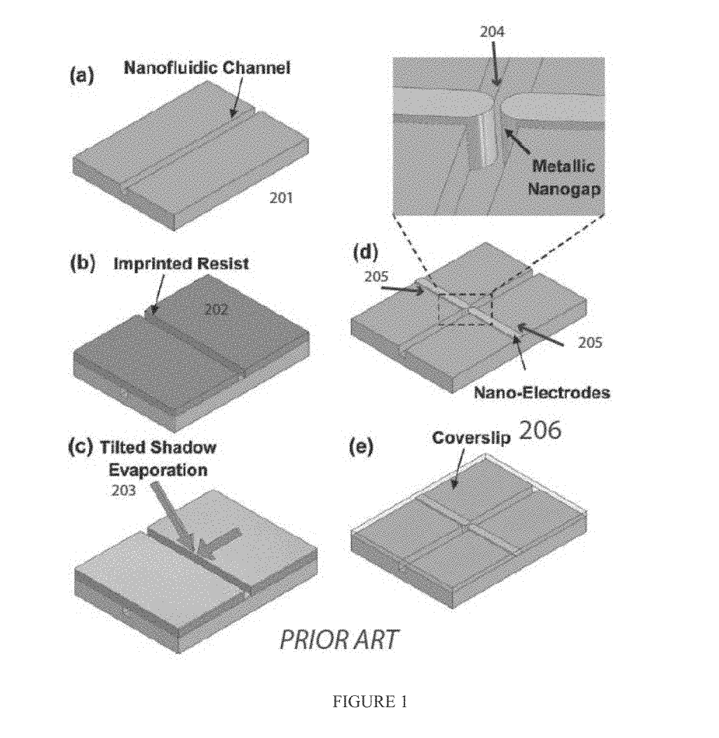

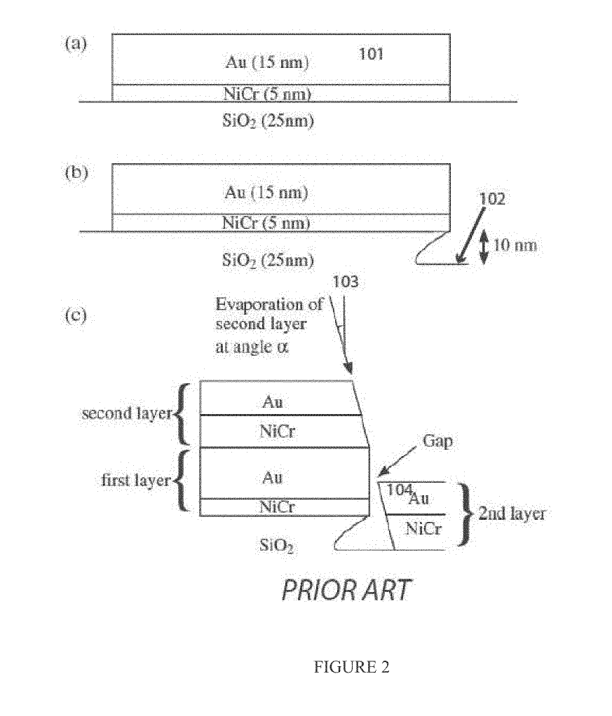

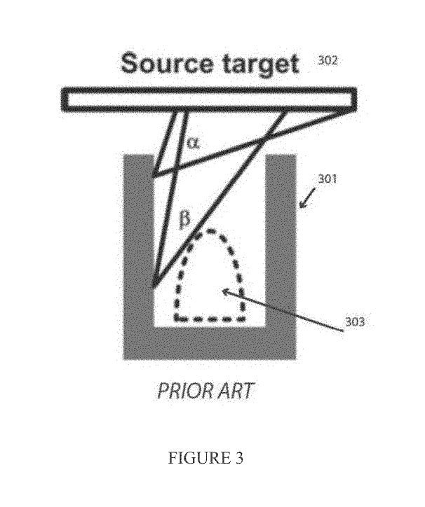

### DESCRIPTION OF SOME OF THE EMBODIMENTS OF THE PRESENT DISCLOSURE

According to some embodiments, a channel and reading gap formed by evaporation steps (e.g., alone), is provided, such that the formation results in the reading gap and channel being automatically aligned with each other.

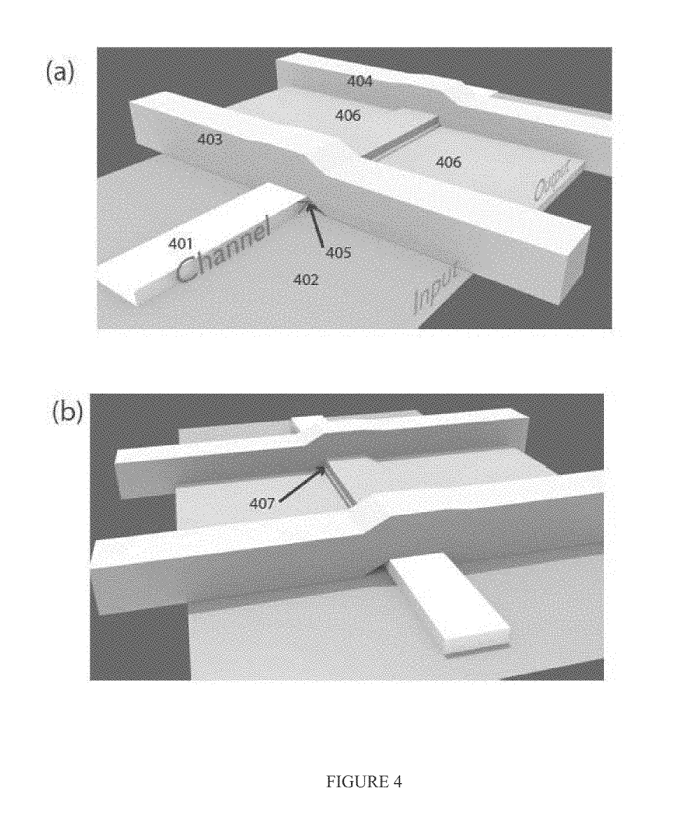

An outline of a device according to some embodiments is shown from two views in FIG. 4. Accordingly, a strip of a dielectric material (such as oxides of silicon, aluminum, hafnium etc.) 401 is formed by lithography on the surface of a substrate, 402. The substrate may be Si or SiO₂ or SiN or other similar material. A second set of strips (403, 404) may be formed by evaporating further dielectric through a lithographically defined mask at an angle. The deposition of material at an angle (other than perpendicular) results in a channel 405 formed by the shadow of the first strip 401. In some embodiments, if the first strip 401 is 20 nm high (for example) and the angle of evaporation of the second strip occurs at an angle of 45 degrees (for example) to the normal to the surface 402, then the channel is approximately a right triangle with a height and base of 20 nm and hypotenuse of about 28 nm (for example). A third mask is then used to deposit metal 406, also at an angle that is not normal to the surface. The metal is gold (Au) in the preferred embodiment, but other noble metals such as Pt or Pd may be used. If, as in the previous example, the thickness of the first strip 401 is 20 nm (for example), and a 19 nm (for example) thick layer of metal 406 is deposited, then the gap between the upper and lower metal layers will be 1 nm. FIG. 4b shows a view looking into the other side of the strip 403. The gap between metal electrodes is shown 407 where it touches the exit of the nanochannel under strip 403. The second strip 404 serves as a fluid barrier as will be described below.

Cross sections (Side View) and plans (Top View) of the fabrication process according to some embodiments are shown in FIG. 5 (a, b, c side views, d, e, f the corresponding top views). The first strip deposited is 501 on the substrate 502. The second (perpendicular) strip is 503. It forms a bridge 504 over the step, enclosing a channel 505 in the shadow of the first strip 501. The integrity of the bridge 504 may be enhanced by rocking the device over a small (5 to 15 degrees, for example) angle as the deposition of the strip 503 occurs.

Three perpendicular strips are shown (503 in FIG. 5e). These serve as barriers to define two separate fluid reservoirs (508 and 509 in FIG. 5f), according to some embodiments.

Note that imperfect masking and alignment may leave continuous metal contacting the upper and lower electrodes along the tops of the strips 503. This may be readily removed by physically delaminating the metal in these regions by pressing the device against an adhesive surface that touches the tops of the strips 503 but not the main electrode pads 506.

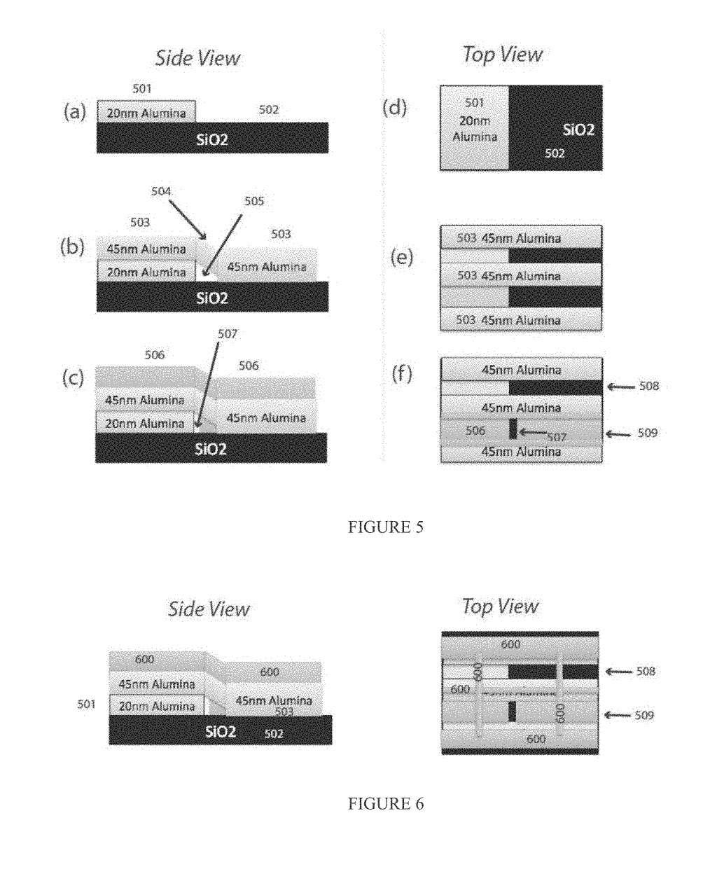

The fluid reservoirs 508 and 509 may be sealed using a silicone rubber gasket shown as 600 in FIG. 6. The fluid reservoirs may be filled with electrolyte solution and reference electrodes are placed into each of the reservoirs 508 and 509. In FIG. 6, the input reservoir is shown as 508. If a negatively charged polymer such as DNA is placed into the electrolyte in the input reservoir 508, and the reference electrode in the output reservoir 509 biased to be positive with respect to the reference electrode in the input reservoir, the DNA molecules will be driven into the channel 405 on the input side (referring to FIG. 4a) and forced to emerge into the nm-sized gap 407 that connects to the output reservoir (FIG. 4b).

The structure we have described is self-aligning and readily fabricated to force single-stranded DNA molecules through gaps as small as 1-2 nm in extent.

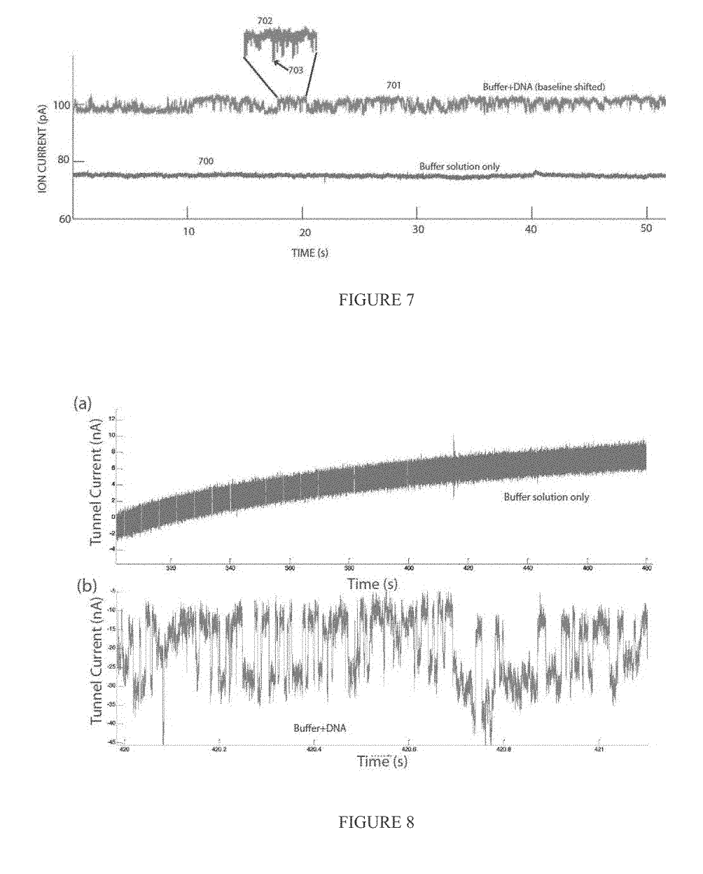

The operation of the nanochannel according to some embodiments is illustrated in FIG. 7. As shown (bottom trace), an ion current passing from reservoir 508 to reservoir 509 when a 1 mM sodium phosphate buffer (pH7) is place in both reservoirs and a bias of +300 mV applied to the reference electrode in reservoir 509 with respect to the reference electrode in the input reservoir 508. The ion current 700, according to some embodiments, is constant with time. In one example, when a 30 nt long single stranded DNA fragment was added to the input reservoir to a concentration of approximately one micromolar, the resulting current trace 701 shows features that change with time (note that the current is still about 75 pA, but the trace 701 has been shifted upwards for clarity). An expanded portion 702 of the trace shows the dips in current that are expected when DNA molecules block the nanopore (407 in FIG. 4b) formed at the exit of the nanochannel by the metal gap.

Corresponding traces of a tunnel current that passes between the electrodes 506 across the nanogap (507 in FIG. 5f) are shown in FIG. 8. The bias applied across the tunneling gap was 300 mV in this case. FIG. 8a shows a current trace in buffer solution alone. While there is some drift owing to mechanical drift in the dimension of the tunnel gap, the current is featureless. In this example, the electrodes were functionalized prior to this measurements with the reader molecule 4(5)-(2-mercaptoethyl)-1H imideazole-2-carboxamide as described in the '386 publication and Pang et al. (Pang, Ashcroft et al. 2014).

When DNA is added to the input reservoir 508, large jumps in current are seen (FIG. 8b) characteristic of electron tunneling through DNA bases via the reader molecules (see for example Pang et al. (Pang, Ashcroft et al. 2014)).

This exemplary data produced according to some embodiments of the present disclosure illustrate the goal of fabricating a tunnel junction aligned with a nanochannel in such a way as to force DNA through the tunnel junction. Furthermore, in some embodiments, no critical alignment steps are required to fabricate these devices.

While various inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be an example and that the actual parameters, dimensions, materials, and/or configurations will depend upon the specific application or applications for which the inventive teachings is/are used. Those skilled in the art will recognize, or be able to ascertain using no more than routine experimentation, many equivalents to the specific inventive embodiments described herein. It is, therefore, to be understood that the foregoing embodiments are presented by way of example only and that, within the scope of the appended claims and equivalents thereto, inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure. Moreover, some embodiments are distinguishable from the prior art by lack of or elimination of structure, functionality and/or a step specifically disclosed in the prior art (e.g., some embodiments may be claimed with negative limitations to distinguish them from the prior art).

Also, various inventive concepts may be embodied as one or more methods, of which an example has been provided. The acts performed as part of the method may be ordered in any suitable way. Accordingly, embodiments may be constructed in which acts are performed in an order different than illustrated, which may include performing some acts simultaneously, even though shown as sequential acts in illustrative embodiments.

Any and all references to publications or other documents, including but not limited to, patents, patent applications, articles, webpages, books, etc., presented anywhere in the present application, are herein incorporated by reference in their entirety. Moreover, all definitions, as defined and used herein, should be understood to control over dictionary definitions, definitions in documents incorporated by reference, and/or ordinary meanings of the defined terms. In this regard, references to publications in the detailed description are included to provide, at least for some embodiments, a supporting and enabling disclosure, as well providing additional disclosure that when combined with one and/or another disclosed inventive subject matter provide yet additional embodiments.

### LITERATURE CITED

Liang, X. and S. Y. Chou (2008). “Nanogap Detector Inside Nanofluidic Channel for Fast Real-Time Label-Free DNA Analysis.” Nano Lett. 8: 1472-1476.
Pang, P., B. Ashcroft, et al. (2014). “Fixed Gap Tunnel Junction for Reading DNA Nucleotides.” ACS Nano: Published online November 7. DOI: 10.1021/nn505356g.
Perry, J. L. and S. G. Kandlikar (2006). “Review of fabrication of nanochannels for single phase liquid flow.” Microfluidics and Nanofluidics 2: 185-193.
Sun, L. F., S. N. Chin, et al. (2005). “Shadow-evaporated nanometre-sized gaps and their use in electrical studies of nanocrystals.” Nanotechnology 16: 631-634.
Liang, X. and S. Y. Chou (2008). “Nanogap Detector Inside Nanofluidic Channel for Fast Real-Time Label-Free DNA Analysis.” Nano Lett. 8: 1472-1476.
Pang, P., B. Ashcroft, et al. (2014). “Fixed Gap Tunnel Junction for Reading DNA Nucleotides.” ACS Nano: Published online November 7. DOI: 10.1021/nn505356g.
Perry, J. L. and S. G. Kandlikar (2006). “Review of fabrication of nanochannels for single phase liquid flow.” Microfluidics and Nanofluidics 2: 185-193.
Sun, L. F., S. N. Chin, et al. (2005). “Shadow-evaporated nanometre-sized gaps and their use in electrical studies of nanocrystals.” Nanotechnology 16: 631-634.

What is claimed is:

1. A method for making a device having a nanogap and a nanochannel aligned with each other, the method comprising:
depositing a first dielectric strip onto a first surface of a substrate;
depositing a plurality of dielectric strips onto the first dielectric strip and the first surface at a first angle with respect to the first surface, wherein the first angle is not 90°, and wherein the plurality of dielectric strips comprises a second dielectric strip, a third dielectric strip, and a fourth dielectric strip that are substantially parallel to each other, the third dielectric strip positioned in between the second fourth dielectric strips, thereby forming the nanochannel bound by the first dielectric strip, the first surface, and the third dielectric strip; and
depositing a metallic layer onto an area bound by the second and third dielectric strips at a second angle with respect to the first surface, wherein the second angle is not 90°, thereby forming a first sensing electrode on the first dielectric strip, a second sensing electrode on the first surface, and the nanogap separating the first and second sensing electrodes.

2. The method of claim 1, wherein the first dielectric strip comprises a silicon oxide, an aluminum oxide, or a hafnium oxide.

3. The method of claim 1, wherein the plurality of dielectric strips comprises a silicon oxide, an aluminum oxide, or a hafnium oxide.

4. The method of claim 1, wherein the metallic layer comprises Au, Pt, or Pd.

5. The method of claim 1, wherein the substrate comprises Si, SiO₂, or SiN.

6. The method of claim 1, wherein the metallic layer has a thickness less than that of the first dielectric strip.

7. The method of claim 1, wherein the plurality of dielectric strips is perpendicular to the first dielectric strip.

8. The method of claim 1, wherein the first dielectric strip is 20 nm thick.

9. The method of claim 8, wherein the metallic layer is 19 nm thick.

10. The method of claim 1, wherein the plurality of dielectric strips is 45 nm high thick.

11. The method of claim 1, wherein the first angle is 45°.

12. The method of claim 1, wherein the nanogap is 1 nm.
